(19) United States
(12) Patent Application Publication
(10) Pub. No.: US 2022/0105081 A1
(43) Pub. Date: Apr. 7, 2022

(54) OPIOID ANTAGONIST COMPOUNDS AND METHODS OF TREATING MEDICAL CONDITIONS

(71) Applicant: VDM Biochemicals, Inc., Lake Forest, CA (US)

(72) Inventor: Vardan Martirosyan, Lake Forest, CA (US)

(73) Assignee: VDM Biochemicals, Inc., Lake Forest, CA (US)

(21) Appl. No.: 16/948,922

(22) Filed: Oct. 6, 2020

(51) Int. Cl. A61K 31/4468; A61K 47/12; A61P 25/36; A61P 25/18; A61P 25/16; A61P 25/06; A61P 25/24; A61P 25/22

(52) U.S. Cl. CPC A61K 31/4468; A61K 47/12; A61P 25/36; A61P 25/22; A61P 25/16; A61P 25/06; A61P 25/24; A61P 25/18

(57) ABSTRACT

The present invention is aimed at providing an opioid antagonist compound and its medical uses. The medical uses the compound of formula (I) (shown below) include complete or partial reversal of narcotic depression with minimal to no withdrawal symptoms, treatment of opioid addiction, depression, opioid withdrawal side effect treatment, schizophrenia, hypertension, scleroderma, dyskinesia, bipolar disorder, ADHD, addictive behaviors, Parkinson's disease, eating disorders, anorexia nervosa, migraine, and memory disorders.

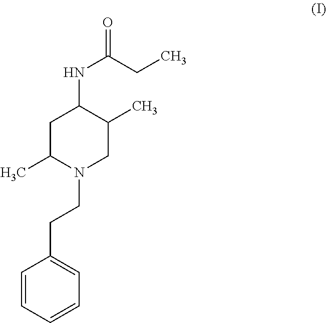 [SMILES: CCC(=O)NC1CC(C)N(CCc2ccccc2)CC1C]

### BACKGROUND

Opioid antagonist compounds have been approved by the FDA for some time for the complete or partial reversal of opioid intoxication. For example, naloxone was approved by the FDA in 1971 and first marketed as Narcan® injection for the complete or partial reversal of opioid intoxication. It has subsequently become a multisource prescription generic drug available in two strengths, 0.4 mg/mL and 1.0 mg/mL.

At present, naloxone is utilized by emergency services personnel for reversal of suspected opioid overdose. Hospital emergency departments carry this medication for the same purpose. Naloxone is also indicated as a reversal agent when the effects of therapeutic use of opioids are no longer medically necessary.

According to the Centers for Disease Control and Prevention, in 2008, poisoning became the leading cause of injury death in the United States. Nearly 9 out of 10 poisoning deaths are caused by drugs. During the past three decades, drug poisoning deaths increased by six fold, from about 6,100 in 1980 to about 36,500 in 2008. Between 1999 and 2008, the number of drug poisoning deaths involving opioid analgesics more than tripled from about 4,000 to about 14,800. In 2008, opioid analgesics were involved in more than 40% of drug poisonings, whereas in 1999, opioid analgesics were involved in 25% of drug poisoning.

Due to the increasing need for opioid overdose reversal agents, there is a need in the art for new and alternate compositions. Although naloxone is generally effective for opioid reversal, as with any medication, there are people who do not react well to naloxone. An alternative medication would be useful. In addition, when administered, naloxone produces opioid withdrawal symptoms. There is unmet need for an opioid reversal agent that does not produce withdrawal symptoms. The instant disclosure seeks to address one or more of these unmet needs.

In addition, opioid antagonist compounds have a role in the treatment of opioid use disorder. According to the Diagnostic and Statistical Manual for Mental Disorders, 5th Edition (DSM-5), opioid use disorder is characterized by signs and symptoms that reflect compulsive, prolonged self-administration of opioid substances that are used for no legitimate medical purpose or, if another medical condition is present that requires opioid treatment, they are used in doses greatly in excess of the amount needed for that medical condition. In a 2015 report from the National Survey on Drug Use and Health, 12.4 million Americans engaged in non-medical use of prescription pain relievers, including opioids. Approximately 2.06 million Americans met criteria for prescription pain reliever use disorder. The same report suggested that 5.1 million people aged 12 and older have used heroin at some point in their lives, with 828,000 using in the past year and 329,000 using in the past month. There were approximately 580,000 people who had a heroin use disorder in the past year. Perhaps most concerning, deaths from overdose of opioid analgesics (including opioids, methadone and other synthetic narcotics) showed a 5.2-fold increase from 5,528 to 28,647 deaths between 2001 and 2015. Similarly, heroin-related overdose fatalities showed a 5.4-fold increase during this same period, from 1,779 deaths in 2001 to 12,989 in 2015. An emerging concern contributing to recent increases in opioid overdose deaths were 9,580 deaths due to synthetic opioids (other than methadone) which increased 72% in one year (since 2014).

Opioid antagonist compounds have a role in the treatment of opioid use disorder, including opioid dependence. In fact, opioid antagonists such as naltrexone are used to treat opioid use disorder. However, use of naltrexone causes withdrawal symptoms, thus the patient must go through a detox protocol and be free from opioids to start naltrexone treatment. The detox can take about a week to ten days in which the patient typically undergoes severe withdrawal side effects. In fact, the detox side effects are so severe, many patients choose against starting or completing opioid use disorder treatment. There is a long felt need for an opioid antagonist compound that does not cause severe withdrawal symptoms, thus not requiring a 7 to 10 day detox before the initiation of treatment of opioid dependence.

The present invention provides a pharmaceutical compound, methods for the preparation thereof, and its medical use. The medical use for the pharmaceutical compound such as the compound of formula (I) (shown below) include complete or partial reversal of narcotic depression, including respiratory depression, induced by opioids including natural and synthetic narcotics, such as heroin, fentanyl, morphine, oxycodone, hydrocodone, propoxyphene, methadone and certain narcotic-antagonist analgesics, such as nalbuphine, pentazocine and butorphanol, as described in U.S. Pat. No. 9,650,338, which is incorporated by reference.

An exemplary embodiment of such a compound that provides a technical solution to the above problems is an opioid antagonist compound comprising a pharmaceutical composition represented by formula (I), an enantiomer, a diastereoisomer and a racemate thereof, and a pharmaceutically acceptable salt or solvate thereof:

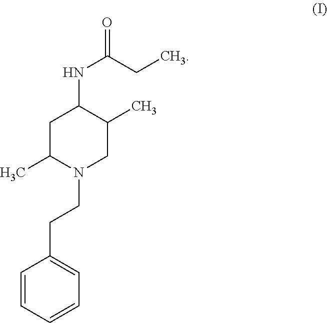 [SMILES: CCC(=O)NC1CC(C)N(CCc2ccccc2)CC1C]

In the opioid antagonist compound of the present invention, the term “pharmaceutically acceptable salt” means a compound represented by formula (I) in salt form that retains at least a portion of, and in certain instances, all of the therapeutic efficacy and nontoxicity of the non-salt form. In certain exemplary embodiments, a salt is formed by contacting a compound of formula (I) with an acid. Examples of such acids include inorganic acids, for example, hydrochloric acid, sulfuric acid, nitric acid, phosphoric acid and the like; or organic acids such as acetic acid, trifluoroacetic acid, propionic acid, glycolic acid, 2-hydroxy propionic acid, 2-oxopropionic acid, oxalic acid, malonic acid, succinic acid, maleic acid, fumaric acid, malic acid, tartaric acid, 2-hydroxy-1,2,3-tricarballylic acid, methyl sulfonic acid, ethyl sulfonic acid, benzene sulfonic acid, 4-methyl benzene sulfonic acid, cyclohexyl sulfinic acids, 2-hydroxy benzoic acid, 4-amino-2-hydroxy benzoic acid, and so on. These salts are well known by the skilled person in the art. In addition, certain salts may be preferred over other salts based on factors, such as solubility, stability, ease of preparation, etc.

An exemplary method for preparing the compound of formula (I) is as follows:

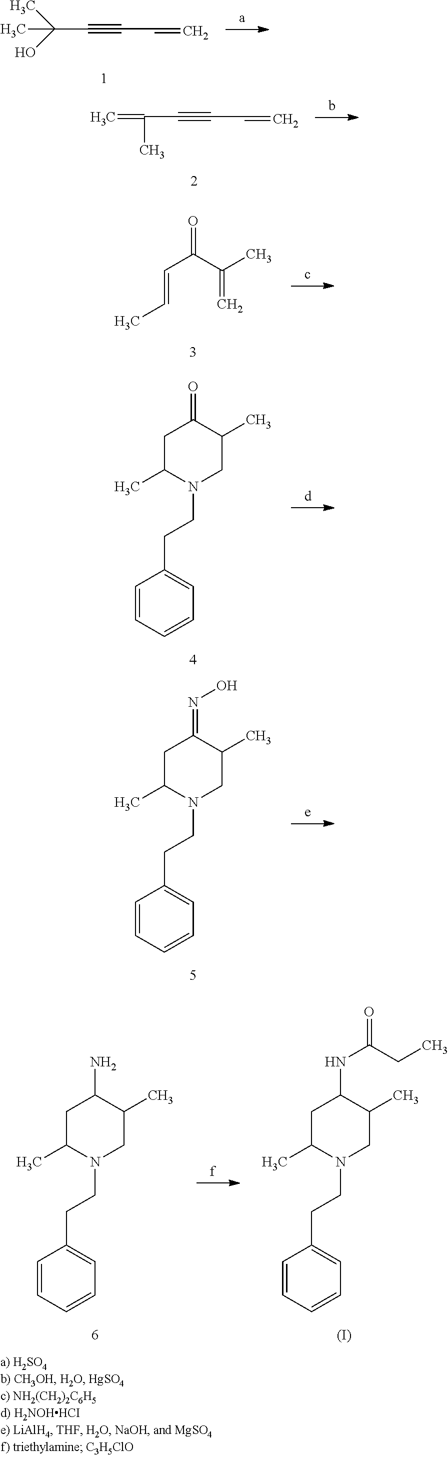

### BRIEF DESCRIPTION

Various details of the present invention are hereinafter summarized to provide a basic understanding. This summary is not an extensive overview of the disclosure and is neither intended to identify certain elements of the disclosure nor to delineate scope thereof. Rather, the primary purpose of this summary is to present some concepts of the disclosure in a simplified form prior to the more detailed description that is presented hereinafter.

In one illustrative embodiment, a pharmaceutical composition for the treatment of a medical condition consisting of opioid addiction, depression, opioid withdrawal side effect treatment, opioid overdose treatment without withdrawal symptoms, schizophrenia, hypertension, scleroderma, dyskinesia, bipolar disorder, ADHD, addictive behaviors, Parkinson's disease, eating disorders, anorexia nervosa, migraine, and memory disorders comprising an effective amount of the compound of formula (I):

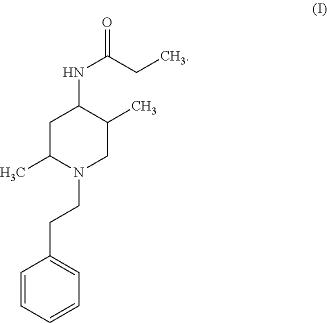 [SMILES: CCC(=O)NC1CC(C)N(CCc2ccccc2)CC1C]

In another embodiment, an injectable pharmaceutical composition for use in treating opioid addiction, depression, anxiety and panic disorders, hypertension, scleroderma, dyskinesia associated with Parkinson's disease, schizophrenia, bipolar disorder, ADHD, addictive behaviors, Parkinson's disease, eating disorders, anorexia nervosa, migraine, opioid overdose without withdrawal effects, and memory disorders, comprising: (a) 1-(2-phenylethyl)-2,5-dimethyl-4-propionil aminopiperidin or a pharmaceutically acceptable salt thereof, and (b) a pH adjustor.

In yet another embodiment, pharmaceutical composition for the treatment of opioid use disorder comprising an effective amount of the compound of formula (I):

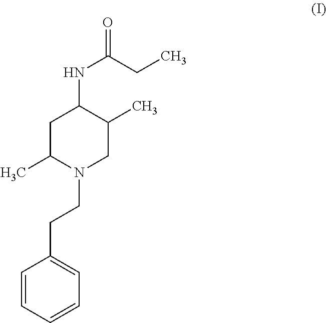 [SMILES: CCC(=O)NC1CC(C)N(CCc2ccccc2)CC1C]

wherein said treatment causes minimal withdrawal symptoms.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 shows comparative graphs showing morphine withdrawal signs in VDM-001 treated versus Naloxone treated morphine dependent rats.

FIG. 2 is a graph showing morphine plasma levels were not different in VDM-001 treated versus Naloxone treated morphine dependent rats in the study of FIG. 1.

FIG. 3 shows the percent agonist or antagonist responses of VDM-001 to the major protein targets detected in the DiscoverX GPCR screen.

FIG. 4 shows comparative graphs showing VDM-001 versus naltrexone and naloxone.

### DETAILED DESCRIPTION

The present disclosure may be understood more readily by reference to the following detailed description of desired embodiments and the examples included therein. In the following specification and the claims which follow, reference will be made to a number of terms which shall be defined to have the following meanings.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

The term “comprising” is used herein as requiring the presence of the named components/steps and allowing the presence of other components/steps. The term “comprising” should be construed to include the term “consisting of”, which allows the presence of only the named components/steps.

Numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values). The endpoints of the ranges and any values disclosed herein are not limited to the precise range or value; they are sufficiently imprecise to include values approximating these ranges and/or values.

The modifier “about” used in connection with a quantity is inclusive of the stated value and has the meaning dictated by the context. When used in the context of a range, the modifier “about” should also be considered as disclosing the range defined by the absolute values of the two endpoints. For example, the range of “from about 2 to about 10” also discloses the range “from 2 to 10.” The term “about” may refer to plus or minus 10% of the indicated number. For example, “about 10%” may indicate a range of 9% to 11%, and “about 1” may mean from 0.9-1.1.

The term “active ingredient” or “pharmaceutically active compound” is defined in the context of a “pharmaceutical composition” and is intended to mean a component of a pharmaceutical composition that provides the primary pharmacological effect, as opposed to an “inactive ingredient” which would generally be recognized as providing little or no pharmaceutical benefit apart from, for example, formulation or delivery.

The term “agonist,” as used herein, refers to a moiety that interacts with and activates a receptor, and thereby initiates a physiological or pharmacological response characteristic of that receptor. The term “antagonist,” as used herein, refers to a moiety that competitively binds to a receptor at the same site as an agonist, but which does not activate the intracellular response initiated by the active form of the receptor and can thereby inhibit the intracellular responses by an agonist or partial agonist. An antagonist does not diminish the baseline intracellular response in the absence of an agonist or partial agonist. The term “inverse agonist” refers to a moiety that binds to the endogenous form of the receptor or to the constitutively activated form of the receptor and which inhibits the baseline intracellular response initiated by the active form of the receptor below the normal base level of activity which is observed in the absence of an agonist or partial agonist.

The term “naloxone,” as used herein, refers to a compound of the following structure:

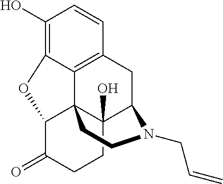

Other names for naloxone include: 17-allyl-4,5a-epoxy-3,14-dihydroxymorphinan-6-one; (−)-17-allyl-4,5a-epoxy-3,14-dihydroxymorphinan-6-one; 4,5a-epoxy-3,14-dihydroxy-17-(2-propenyl)morphinan-6-one; and (−)-12-allyl-7,7a,8,9-tetrahydro-3,7a-dihydroxy-4aH-8,9c-iminoethanophenanthro[4,5-bcd]furan-5(6H)-one.

The term “opioid intoxication,” as used herein, refers to an acute medical condition induced by use of one or more opioids. The term “opioid overdose,” as used herein, refers to an acute medical condition induced by excessive use of one or more opioids. Symptoms of opioid intoxication and opioid overdose include respiratory depression (including postoperative opioid respiratory depression, acute lung injury, and aspiration pneumonia), central nervous system depression (which may include sedation, altered level consciousness, miotic (constricted) pupils), and cardiovascular depression (which may include hypoxemia and hypotension). Visible signs of opioid intoxication, opioid overdose or suspected opioid overdose include: unresponsiveness and/or loss of consciousness; slow, erratic, or stopped breathing; slow, erratic, or stopped pulse; deep snoring or choking/gurgling sounds; blue or purple fingernails or lips; pale and/or clammy face; slack or limp muscle tone; contracted pupils; and vomiting. Because opioid overdose may be difficult to diagnose or quantify, or both, particularly by a lay person, as used herein, treatment of opioid overdose includes treatment of suspected opioid overdose in opioid-intoxicated patients. Opioids that cause opioid intoxication and may induce opioid overdose include, codeine, morphine, methadone, fentanyl, oxycodone.HCl, hydrocodone bitartrate, hydromorphone, oxymorphone, meperidine, propoxyphene, opium, heroin, tramadol, tapentadol, and certain narcotic-antagonist analgesics, such as, nalbuphine, pentazocine and butorphanol.

The term “pharmaceutical composition,” as used herein, refers to a composition comprising at least one active ingredient; including but not limited to its enantiomers, diastereomers, racemates, salts, solvates and hydrates, whereby the composition is amenable to use for a specified, efficacious outcome in a mammal (for example, without limitation, a human).

“Pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, oxalic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

“Treating” or “treatment” of a disease includes: (1) preventing the disease, i.e., causing the clinical symptoms of the disease not to develop in a mammal that may be exposed to or predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

Utility: The compounds of the invention have a variety of physiological properties. In particular, the present inventors have discovered that compounds of the invention can modulate a variety of receptors including, but not limited to, μ-, κ and ORL-1 opioid receptors, sigma receptors, Dopamine D4 receptor, Serotonin 1A receptor and Serotonin 1B receptor, adrenoreceptors in particular α2B-adrenoreceptor. In particular, the compound of the invention is found to be antagonists or partial agonist of these receptors. Accordingly, any clinical conditions that are due or associated with at least in part to abnormal activation of one or more of these receptors can be treated by the compounds of the invention.

### Opioid Antagonist Compounds

Opioid receptor antagonist compounds are a well recognized class of chemical agents. They have been described in detail in the scientific and patent literature. Opioid antagonist compounds, such as naloxone and the compound of formula (I) of the present invention, namely, N-[2,5-dimethyl-1-(2-phenylethyl)piperidin-4-yl]propanamide or 2,5-Dimethyl-1-(2-phenyl)ethyl-4-propionilaminopiperidin, are agents which specifically reverse the effects of opioid agonist compounds but have no opioid agonist activity. Naltrexone is an opioid antagonist compound that is also a partial inverse agonist compound. It is well known that opioid antagonist compounds such as Naltrexone and Naloxone have a common side effect, major withdrawal symptoms.

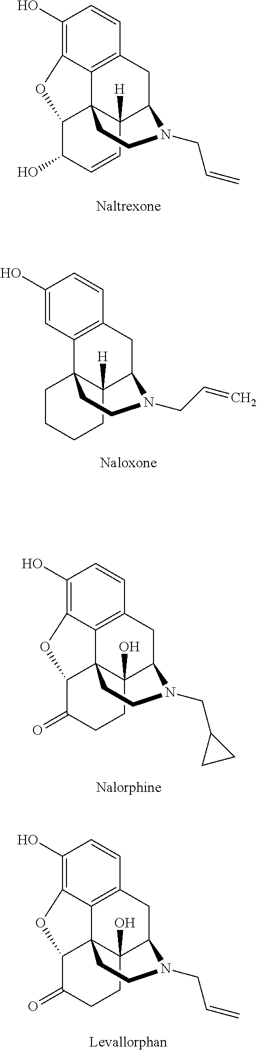

There are also known opioid receptor antagonist compounds that are also partial opioid receptor agonist compounds. Examples include Nalorphine and Levallorphan.

The structure of the known opioid receptor antagonist compounds and the opioid antagonist/partial agonist compounds all share the same basic structure. One of ordinary skill in the art would not expect a compound lacking the fused ring central structure of the compounds above to show opioid antagonist activity. However, it was surprisingly discovered that the pharmaceutical composition of formula (I) displayed such activity.

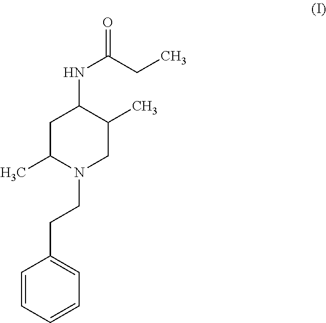 [SMILES: CCC(=O)NC1CC(C)N(CCc2ccccc2)CC1C]

The pharmaceutical composition of formula (I) is also known as 1-(2-phenylethyl)-2,5-dimethyl-4-propionil aminopiperidin or N-[2,5-dimethyl-1-(2-phenylethyl)piperidin-4-yl]propanamide or 2,5-Dimethyl-1-(2-phenyl)ethyl-4-propionilaminopiperidin. The pharmaceutical composition of formula (I) functions as an opioid receptor antagonist compound, even though it is quite different in structure to known opioid antagonist compounds. In fact, the pharmaceutical composition of formula (I) of the present invention actually more closely resembles an opioid agonist, fentanyl, shown below:

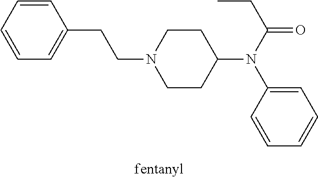 [SMILES: CCC(=O)N(c1ccccc1)C1CCN(CCc2ccccc2)CC1]

Fentanyl, in stark contrast to the pharmaceutical composition of formula (I), is a potent opioid agonist compound. In other words, it is the functional opposite of the inventive pharmaceutical composition of formula (I). Fentanyl is a potent analgesic that has historically been used to treat breakthrough pain. The abuse of fentanyl has led to many opioid overdoses, which are actually treated by opioid antagonist compounds such as the pharmaceutical composition of formula (I). Considering that the structure of the pharmaceutical composition of formula (I) much more closely resembles a known powerful opioid agonist than the known opioid antagonists, it was a surprising discovery that the inventive compound of formula (I) functions as an opioid receptor antagonist. According to the CDC, Fentanyl is estimated to be 80 times as potent as morphine and hundreds of times more potent than heroin.

The inventive pharmaceutical composition of formula (I) fills the need of a new compound to treat various medical conditions such as to treat opioid overdose without causing withdrawal symptoms, opioid use disorder, opioid addiction, depression, such as major depression, opioid withdrawal side effect treatment, schizophrenia, hypertension, scleroderma, dyskinesia, bipolar disorder, ADHD, addictive behaviors, Parkinson's disease, eating disorders, anorexia nervosa, migraine, and memory disorders.

The invention relates to a pharmaceutical composition for the treatment of individuals with medical conditions that may benefit from the use of the composition of formula (I) such as the pharmaceutical composition of formula (I).

The invention also relates to methods for the treatment of a medical condition comprising administering an effective amount of the composition of formula (I) to an individual in need thereof wherein the medical condition comprises opioid addiction, depression, opioid withdrawal side effect treatment, schizophrenia, hypertension, scleroderma, dyskinesia, bipolar disorder, ADHD, addictive behaviors, Parkinson's disease, eating disorders, anorexia nervosa, migraine, and memory disorders.

This invention also relates to a method of treating opioid use disorder, opioid intoxication, opioid overdose, or suspected opioid overdose, in an individual in need, comprising administering to said individual an amount of the compound of formula (I) that is effective in treating said opioid intoxication, opioid overdoes or suspected opioid overdose wherein said treatment causes minimal or no withdrawal symptoms. The opioid intoxication or opioid overdose is treated by complete or partial reversal of narcotic depression, including respiratory depression induced by opioids including natural and synthetic narcotics, propoxyphene, methadone, fentanyl, heroin or any similar narcotic. The narcotic depression may also be induced by narcotic-antagonist analgesic compounds such as nalbuphine, pentazocine, butorphanol, or similar analgesic medication.

The following examples are provided to further illustrate the invention.

### Example 1

Morphine Dependent Rats Dosed with Naloxone and Composition of Formula I (VDM-001).

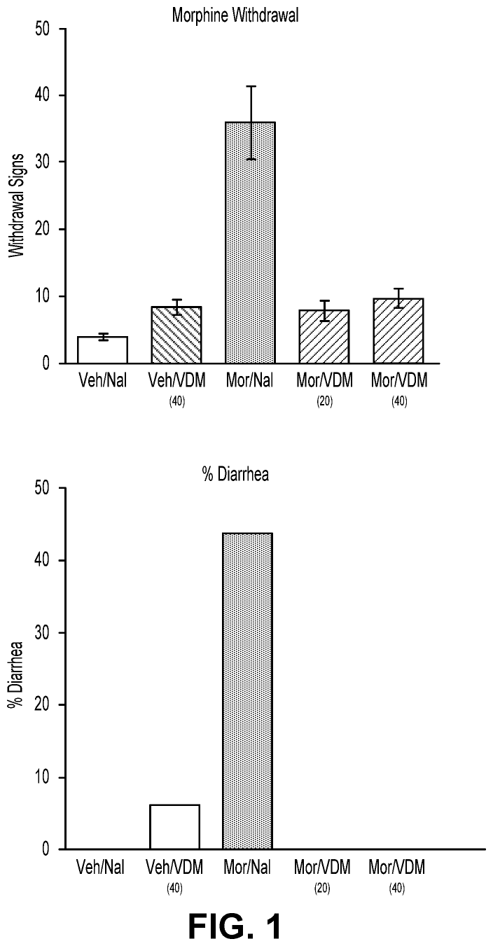

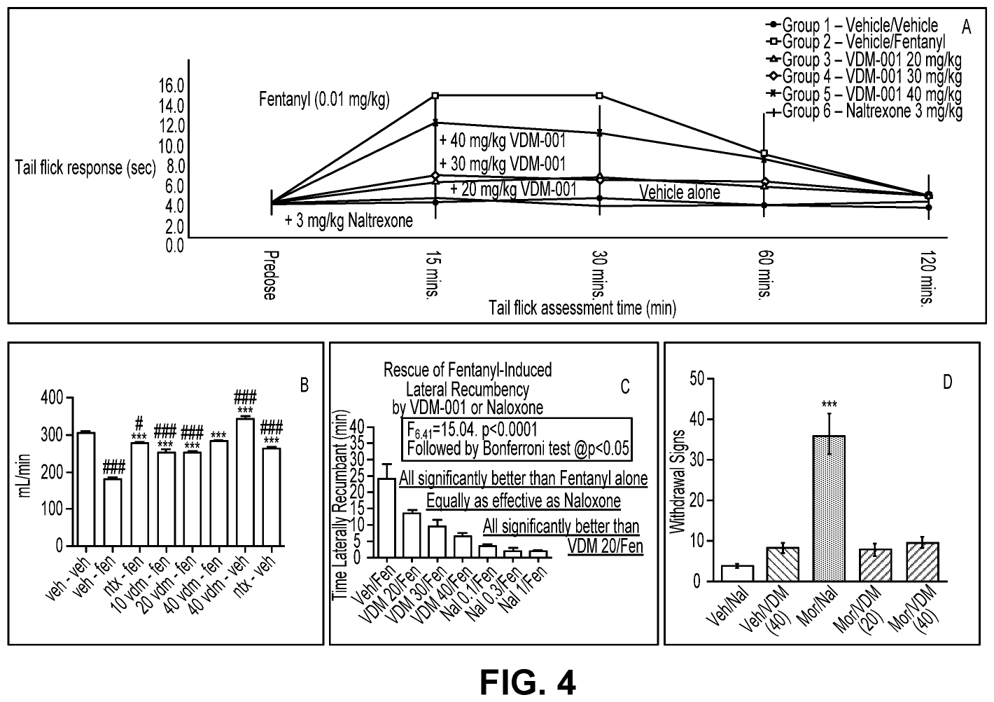

The effects of the inventive pharmaceutical composition of formula (I) have been compared to naloxone and are set forth below. In Example 1, 40 morphine-dependent rats were dosed with naloxone (1 mg/kg) and the composition of formula (I) (also known as VDM-001) (20 or 40 mg/kg). FIG. 1 and FIG. 4D show the resultant opiate withdrawal signs (number of rats exhibiting withdrawal symptoms out of 40) in morphine-dependent rats when dosed with naloxone (1 mg/kg) vs. the compound of formula (I), i.e. VDM-001 (20 or 40 mg/kg). In addition, FIG. 1 shows incidence of diarrhea, which is a major symptom of withdrawal, was significantly lower for VMD-001 at 40 mg/kg (over 80% for naloxone and less than 20% for VDM-001 40 mg/kg) and equal to the control at VDM-001 20 mg/kg (around 1%). As is clear, large opiate withdrawal signs were shown in morphine-dependent rats dosed with naloxone (1 mg/kg). In stark contrast, the morphine-dependent rats dosed with the compound of formula (I), i.e. VDM-001 (20 or 40 mg/kg) showed minimal, if any, opiate withdrawal signs. Accordingly, VDM-001 is novel in blocking opiates, without inducing withdrawal symptoms such as muscle aches, restlessness, anxiety, lacrimation (eyes tearing up), runny nose, excessive sweating, inability to sleep, yawning very often, diarrhea, abdominal cramping, goose bumps on the skin, nausea and vomiting, dilated pupils and possibly blurry vision, rapid heartbeat, high blood pressure, and other known symptoms. In Example 1, withdrawal symptoms monitored in rats were a cluster of behaviors expressed by the animal, including elevated startle/hyperreactivity, ptosis (eye closure), hypolocomotion, chewing, and tremors/twitches, which translate into some of the clinical symptoms of opioid withdrawal in humans.

There are no other known drugs that block opiates without inducing withdrawal symptoms.

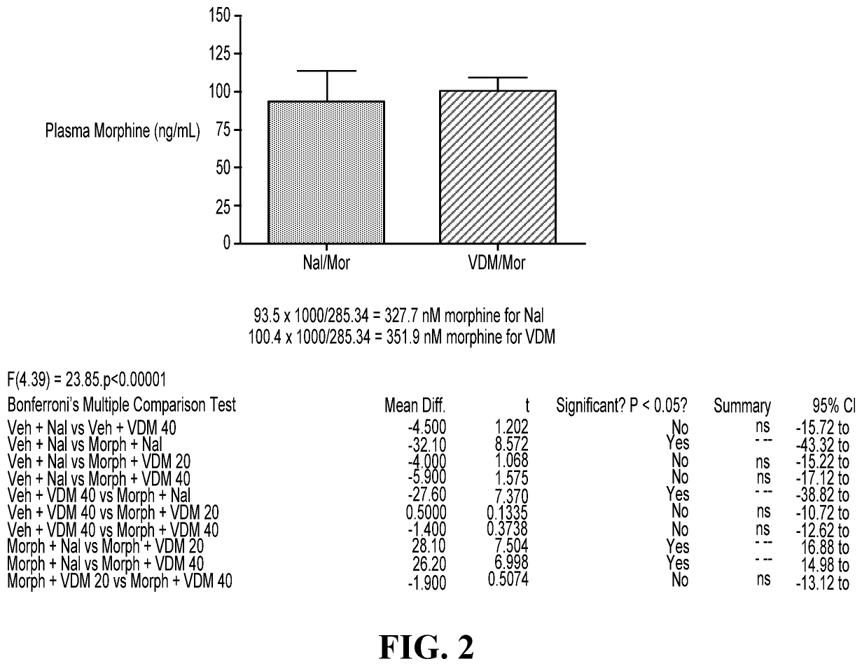

In Example 1, as show in FIG. 2, the rats treated with the composition of formula (I), VDM-001, had similar morphine concentration values to the rats treated with naloxone, thus this was not responsible for the lack of withdrawal symptoms in the rats treated with the compound of formula (I), i.e., VDM-001. Accordingly, VDM-001 at doses known to block the analgesic and respiratory-depressant effects of morphine did not precipitate morphine withdrawal symptoms. This is a unique feature of the compound of formula (I) (VDM-001) that has significant medical value for both use to rescue opioid intoxication/overdose and for the treatment of opioid addiction/opioid use disorder.

### Example 2

DiscoverX Platform Screening of VDM-001 (Composition of Formula (I)).

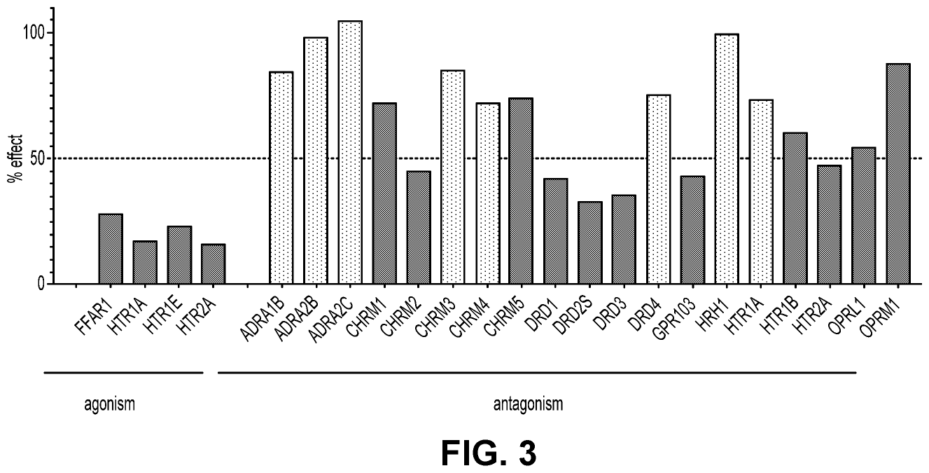

In Example 2, DiscoverX platform screening (GPCR Max Panel) of VDM-001(composition of formula I)) at a concentration of 30 μM (the IC₅₀ for VDM-001 from the GTPgS studies) was conducted to confirm the data from the GTPgS study and to determine broad selectivity of over 336 protein targets. MOR antagonism was confirmed (87% activity) along with other targets with >50%. Overall 13 proteins were engaged by VDM-001 with greater than 50% activity. These proteins and their known functions are described in Table 1 and engagement with VDM-001 is shown in FIG. 3. In this regard, FIG. 3 shows the percent agonist or antagonist responses of VDM-001 to the major protein targets detected in the DiscoverX GPCR screen.

Accordingly, due to the engagement of these proteins In Vitro, the compound of formula (I), VDM-001, could be used to treat any of a number of conditions, such as opioid addiction, depression, anxiety and panic disorders, hypertension, scleroderma, allergic reactions, dyskinesia associated with Parkinson's disease, schizophrenia, bipolar disorder, ADHD, addictive behaviors, Parkinson's disease, eating disorders, anorexia nervosa, migraine, and memory disorders, amongst other conditions, as discussed below.

TABLE 1

| Protein | Activity | Protein Name | Endogenous Ligand | Assay Mode |
|---|---|---|---|---|
| ADRA1B | 84% | Alpha 1B | Acetylcholine | Antagonist |
| | | adrenergic | | |
| | | receptor | | |
| ADRA2B | 98% | Alpha 2B | Acetylcholine | Antagonist |
| | | adrenergic | | |
| | | receptor | | |
| ADRA2C | 104% | Adrenergic | Acetylcholine | Antagonist |
| | | receptor alpha 2C | | |
| HRH1 | 99% | Histamine H1 | Histamine | Antagonist |
| | | receptor | | |
| OPRM1 | 87% | Mu Opiate | Endogenous | Antagonist |
| | | receptor | opioids | |
| OPRL1 | 54% | Nociceptin 1 | Nociceptin | Antagonist |
| | | receptor | | |
| CHRM3 | 85% | Cholinergic | Acetylcholine | Antagonist |
| | | Muscarinic | | |
| | | Receptor 3 | | |
| DRD4 | 75% | Dopamine D4 | Dopamine | Antagonist |
| | | receptor | | |
| CHRM5 | 74% | Cholinergic | Acetylcholine | Antagonist |
| | | Muscarinic | | |
| | | Receptor 5 | | |
| HTR1A | 73% | Serotonin 1A | Serotonin | Antagonist |
| | | receptor | | |
| CHRM1 | 72% | Cholinergic | Acetylcholine | Antagonist |
| | | Muscarinic | | |
| | | Receptor 1 | | |
| CHRM4 | 72% | Cholinergic | Acetylcholine | Antagonist |
| | | Muscarinic | | |
| | | Receptor 4 | | |
| HTR1B | 60% | Serotonin 1B | Serotonin | Antagonist |
| | | receptor | | |

With respect to activity related to proteins ADRA1B, ADRA2B, and ADRA2C, similar to the action of the opioid antagonists, antagonists of the adrenoreceptors bind to the adrenoreceptors and act to inhibit the action of those receptors. Generally, alpha-blockers have shown to be effective in the treatment of various medical conditions including anxiety and panic disorders, hypertension, scleroderma and in the treatment of dyskinesia associated with Parkinson's disease. Thus, the compound of formula (I) could be used to treat these and related conditions.

With respect to protein HRH1 activity, H1 receptors are involved in peripheral effects of histamine where antagonists are commonly used for allergic reactions (antihistamines). In the CNS, the role of H1 receptors is thought to be a driver of weight gain and sedative-like effects. H1 antagonism is not an uncommon activity in a host of CNS drugs. Thus, the compound of formula (I) could be used to treat these and related conditions.

With respect to protein OPRM1 activity, OPRM1 is the main physiological target for most clinically important opioid analgesics. OPRM1-mediated inhibition of voltage-gated calcium channels on central presynaptic terminals of primary afferent nociceptors is thought to be one of the primary mechanisms mediating analgesia at the spinal level. Thus, the compound of formula (I) could be used to treat these and related conditions.

With respect to protein OPRL1 activity, this receptor is involved in the regulation of numerous brain activities. Antagonists targeting NOP are under investigation for their role as treatments for depression, Parkinson's disease, cognitive impairment and obesity. Thus, the compound of formula (I) could be used to treat these and related conditions.

With respect to protein CHRM3 activity, the M3 receptor antagonism can function to block exocrine gland secretions. It also appears to be involved in food intake and weight gain. In addition, this receptor activity could be useful in antidepressant potential. Thus, the compound of formula (I) could be used to treat these and related conditions.

With respect to protein DRD4 activity, as with other dopamine receptor subtypes, the D4 receptor is activated by the neurotransmitter dopamine. It is linked to many neurological and psychiatric conditions including schizophrenia and bipolar disorder, ADHD, addictive behaviors, Parkinson's disease, and eating disorders such as anorexia nervosa. Thus, the compound of formula (I) could be used to treat these and related conditions.

With respect to protein CHRM5 activity, the M5 receptor antagonism can function to modify blood vessel tone. M5 receptors might also function to modulate dopamine efflux. Therapeutic potential can include vascular disorders like migraine and substance abuse disorder. Thus, the compound of formula (I) could be used to treat these and related conditions.

With respect to protein HTR1A activity, this mechanism has been associated with a host of functions suggesting therapeutic utility. Therapeutic potential can include cognition, anxiety and schizophrenia. Thus, the compound of formula (I) could be used to treat these and related conditions.

With respect to protein CHRM1 activity, CHRM1 is involved in salivation, memory and depression. Thus, the compound of formula (I) could be used to treat these and related conditions.

With respect to protein CHRM4 activity, CHRM4 is involved in regulation of dopamine efflux. Thus, the compound of formula (I) could be used to treat conditions related to dopamine and related conditions.

With respect to protein HTR1B activity, HTR1B is a involved in the treatment of depression and schizophrenia. Thus, the compound of formula (I) could be used to treat these and related conditions.

In view of Example 2 and the data of Table 1 and FIG. 3, and due to the engagement of these proteins as shown by the compound of formula (I), VDM-001, the compound of formula (I), i.e. VDM-001, can be used as a pharmaceutical active ingredient to treat any of a number of conditions, such as opioid addiction, depression, anxiety and panic disorders, hypertension, scleroderma, allergic reactions, dyskinesia associated with Parkinson's disease, schizophrenia, bipolar disorder, ADHD, addictive behaviors, Parkinson's disease, eating disorders, anorexia nervosa, migraine, and memory disorders, amongst other conditions.

In addition, the compound of formula (I), also known as VDM-001, is a partial agonist at mu opioid receptors, an antagonist at kappa and ORL1 receptors and has either agonist or antagonist activity at Sigma sites. In addition, because VDM-001 reacts with different receptors in different ways than the known opioid agonists and antagonists, new therapeutic treatments not typically associated with opioid agonist and antagonist are a new discovery.

### Examples 3 and 4

Comparison of VDM-001 with Naltrexone.

In Example 3, as shown in FIG. 4A-4B, studies were conducted to evaluate the potential of VDM-001 to modify Fentanyl-induced analgesia. Example 3 shows VDM-001 is an antagonist of the pharmacological effects of fentanyl without inducing withdrawal symptoms in rats, unlike in other known opiate antagonists. FIG. 4A shows the time taken (reaction time) for a rat to flick its tail away from a nociceptive thermal stimulus (an infrared beam of a pre-determined intensity). As shown in FIG. 4A, VDM-001 blocks fentanyl-induced analgesia in rats. It also shows that VDM-001 at 20 mg/kg of VDM-001 has similar blocking effects to 3 mg/kg naltrexone. Comparisons were made to pre-dose values and/or a vehicle treated group. A comparator group using Naltrexone, a known opioid antagonist, was also included. On the day of dosing, tail flick assessments were performed pre-dose to allow allocation into treatment groups. Following administration of Treatment 2, animals were assessed for tail flick latency at 15, 30, 60, and 120 minutes post-dose. The study concluded that intraperitoneal administration of 20, 30, or 40 mg/kg VDM-001 modified Fentanyl induced analgesia for up to 30 minutes post dose. The study also concluded that the most efficacious dose of VDM-001 to display no difference, when compared to vehicle control article or Naltrexone, competitive antagonist opioid receptors, was considered to be 20 mg/kg.

In Example 4, blockade of the respiratory depressant effects of fentanyl in rats was studied with VDM-001 head to head with naltrexone. As shown in FIG. 4B, VDM-001 blocks all parameters of the respiratory depressant effects of Fentanyl in rats. VDM-001 produces full blockade on minute volume but partial blockade on tidal volume and respiratory rate.

Example 3 and 4 and FIGS. 4A-4B together show VDM-001 blocks fentanyl-induced analgesia and respiratory effects similar to naltrexone.

### Example 5

Comparison of VDM-001 with Naloxone.

FIG. 4C shows the results of Example 5 wherein VDM-001 does not differ statistically from Naloxone in its ability to rescue Fentanyl-induced lateral recumbency. In this regard, it shows that both VDM-001 and Naloxone significantly reduce the time that rats were recumbent after fentanyl dosing. FIG. 4D shows the results of Examples 1, the testing of morphine dependent rats when dosed with Naloxone (1 mg/kg) compared to VDM-001 (20 mg/kg and 40 mg/kg). As shown in FIG. 4D, Naloxone precipitated a large number of withdrawal signs when morphine dependent rats were doses with Naloxone. In this regard, FIG. 4D shows Naloxone produced a number of biological signs of withdrawal in rats, which would translate to withdrawal in humans. However, in stark contrast, morphine dependent rats dosed with VDM-001 did not show significant withdrawal signs as compared to non-morphine dependent rats dosed with VDM-001, which would also translate to no withdrawal signs in humans. Accordingly, Example 1, taken together with Example 3, 4, and 5, show that VDM-001 is the only known opiate antagonist compound that can block and prevent opiate analgesia, respiratory depression and other known effects, without inducing withdrawal signs.

Opioid antagonist compounds such as naloxone and naltrexone have a role in the treatment of opioid use disorder, including opioid dependence. In fact, opioid antagonists such as naltrexone are used to treat opioid use disorder. However, use of naltrexone causes withdrawal symptoms, thus the patient must go through a detox protocol and be free from opioids to start naltrexone treatment. The detox can take anywhere from about a week to about ten days in which the patient typically undergoes severe withdrawal side effects. In fact, the detox side effects are so severe, many patients choose against starting or completing opioid use disorder treatment. Accordingly, there is a long felt need for an opioid antagonist compound that does not cause severe withdrawal symptoms, thus not requiring a 7 to 10 day detox before the initiation of treatment of opioid dependence. The VDM-001 compound fulfils this need. Accordingly, a patient with opioid use disorder that wants to treat opioid dependency can begin on VDM-001 without undergoing a detox protocol. This may encourage patients to enter rehab and begin treating opioid dependence treatment with VDM-001 when they have been unsuccessful in the past. In addition, VDM-001 could be used as a bridge for the patient to use during the detox period to minimize or eliminate withdrawal symptoms and allow a successful transition to VDM-001 maintenance or other opioid antagonist compound such as naltrexone.

The present invention includes pharmaceutical compositions comprising at least one compound of the invention or a pharmaceutically acceptable salt or solvate thereof, together with at least one pharmaceutically acceptable carrier, and optionally other therapeutic and/or prophylactic ingredients.

Typically, compound of the invention are administered as pharmaceutical formulations including those suitable for oral (including buccal and sub-lingual), rectal, nasal, topical, pulmonary, or parenteral (including intramuscular, intraarterial, intrathecal, subcutaneous and intravenous) administration or in a form suitable for administration by inhalation or insufflation.

A compound of the invention, together with one or more conventional adjuvants, carriers, or diluents, can be placed into the form of pharmaceutical compositions and unit dosages. The pharmaceutical compositions and unit dosage forms can be comprised of conventional ingredients in conventional proportions, with or without additional active compounds or principles, and the unit dosage forms can contain any suitable effective amount of the active ingredient commensurate with the intended daily dosage range to be employed. The pharmaceutical compositions can be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use.

The compound of the invention can be formulated in a wide variety of oral administration dosage forms. The pharmaceutical compositions and dosage forms can comprise a compound or compounds of the invention or pharmaceutically acceptable salts thereof as the active component. The pharmaceutically acceptable carriers can be either solid or liquid. Solid form preparations include powders, tablets, pills, capsules, cachets, suppositories, and dispersible granules. A solid carrier can be one or more substances which can also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. The powders and tablets preferably contain from about one (1) to about seventy (70) percent of the active compound. Suitable carriers include but are not limited to magnesium carbonate, magnesium stearate, talc, sugar, lactose, pectin, dextrin, starch, gelatine, tragacanth, methylcellulose, sodium carboxymethylcellulose, a low melting wax, cocoa butter, and the like. The term “preparation” is intended to include the formulation of the active compound with encapsulating material as carrier, providing a capsule in which the active component, with or without carriers, is surrounded by a carrier, which is in association with it. Similarly, cachets and lozenges are included. Tablets, powders, capsules, pills, cachets, and lozenges can be as solid forms suitable for oral administration.

Other forms suitable for oral administration include liquid form preparations including emulsions, syrups, elixirs, aqueous solutions, aqueous suspensions, or solid form preparations which are intended to be converted shortly before use to liquid form preparations. Emulsions can be prepared in solutions, for example, in aqueous propylene glycol solutions or may contain emulsifying agents, for example, such as lecithin, sorbitan monooleate, or acacia. Aqueous solutions can be prepared by dissolving the active component in water and adding suitable colorants, flavors, stabilizers, and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and can contain, in addition to the active component, colorants, flavors, stabilizers, buffers, artificial and natural sweeteners, dispersants, thickeners, solubilizing agents, and the like.

The compound of the invention can also be formulated for parenteral administration (e.g., by injection, for example bolus injection or continuous infusion) and can be presented in unit dose form in ampoules, pre-filled syringes, small volume infusion or in multi-dose containers with an added preservative. The compositions can take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and can contain formulatory agents such as preserving, wetting, emulsifying or suspending, stabilizing and/or dispersing agents. Alternatively, the active ingredient can be in powder form, obtained by aseptic isolation of sterile solid or by lyophilization from solution for constitution before use with a suitable vehicle, e.g., sterile, pyrogen-free water.

The compounds of the invention can be formulated for topical administration to the epidermis as ointments, creams or lotions, or as a transdermal patch. Ointments and creams can, for example, be formulated with an aqueous or oily base with the addition of suitable thickening and/or gelling agents. Lotions can be formulated with an aqueous or oily base and will in general also contain one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatine and glycerine or sucrose and acacia; and mouthwashes comprising the active ingredient in a suitable liquid carrier.

The compounds of the invention can be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the invention can be formulated for nasal administration. The solutions or suspensions are applied directly to the nasal cavity by conventional means, for example, with a dropper, pipette or spray. The formulations can be provided in a single or multidose form. In the latter case of a dropper or pipette, this can be achieved by the patient administering an appropriate, predetermined volume of the solution or suspension. In the case of a spray, this can be achieved for example by means of a metering atomizing spray pump.

The compound of the invention can be formulated for aerosol administration, particularly to the respiratory tract and including intranasal administration. The compound will generally have a small particle size for example of the order of five (5) microns or less. Such a particle size can be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol can conveniently also contain a surfactant such as lecithin. The dose of drug can be controlled by a metered valve. Alternatively the active ingredients can be provided in a form of a dry powder, for example, a powder mix of the compound in a suitable powder base such as lactose, starch, starch derivatives such as hydroxypropylmethyl cellulose and polyvinylpyrrolidine (PVP). The powder carrier typically forms a gel in the nasal cavity. The powder composition can be presented in unit dose form, for example, in capsules or cartridges of e.g., gelatine or blister packs from which the powder can be administered by means of an inhaler.

When desired, formulations can be prepared with enteric coatings adapted for sustained or controlled release administration of the active ingredient. For example, the compounds of the invention can be formulated in transdermal or subcutaneous drug delivery devices. These delivery systems are advantageous when sustained release of the compound is necessary or desired and when patient compliance with a treatment regimen is crucial. Compounds in transdermal delivery systems are frequently attached to a skin-adhesive solid support. The compound of interest can also be combined with a penetration enhancer, e.g., Azone (1-dodecylazacycloheptan-2-one). Sustained release delivery systems can be inserted subcutaneously into the subdermal layer by surgery or injection. The subdermal implants encapsulate the compound in a lipid soluble membrane, e.g., silicone rubber, or a biodegradable polymer, e.g., polylactic acid.

The pharmaceutical preparations are typically in unit dosage forms. In such form, the preparation is often subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Other suitable pharmaceutical carriers and their formulations are described in Remington: The Science and Practice of Pharmacy 1995, edited by E. W. Martin, Mack Publishing Company, 19th edition, Easton, Pa.

When it is possible that, for use in therapy, therapeutically effective amounts of a compound of Formula I, as well as pharmaceutically acceptable salts thereof, can be administered as the raw chemical, it is possible to present the active ingredient as a pharmaceutical composition. Accordingly, the disclosure further provides pharmaceutical compositions, which include therapeutically effective mounts of compounds of Formula I or pharmaceutically acceptable salts thereof or a prodrug thereof, and one or more pharmaceutically acceptable carriers, diluents, or excipients. When applied to a combination, the term refers to combined amounts of the active ingredients that result in the therapeutic effect, whether administered in combination, serially, or simultaneously. The compounds of Formula I and pharmaceutically acceptable salts thereof, are as described above. The carrier(s), diluent(s), or excipient(s) must be acceptable in the sense of being compatible with the other ingredients of the formulation and not deleterious to the recipient thereof. In accordance with another aspect of the disclosure there is also provided a process for the preparation of a pharmaceutical formulation including admixing a compound of Formula I, or a pharmaceutically acceptable salt thereof or a prodrug thereof, with one or more pharmaceutically acceptable carriers, diluents, or excipients.

The invention also relates to an injectable pharmaceutical composition comprising the pharmaceutical composition of formula (I) or a pharmaceutically acceptable salt thereof and a pH adjustor. The composition can be used to treat opioid addiction, depression, anxiety and panic disorders, hypertension, scleroderma, dyskinesia associated with Parkinson's disease, schizophrenia, bipolar disorder, ADHD, addictive behaviors, Parkinson's disease, eating disorders, anorexia nervosa, migraine, and memory disorders. In one embodiment, the composition contains about 1 mg to about 100 mg of the compound of formula (I) and about 1 mg to about 100 mg of a pH adjustor. In another embodiment, the composition contains about 10 mg of the compound of formula (I) and about 10 mg of a pH adjustor. In still another embodiment, the pH adjustor is citric acid. In an alternative embodiment, any pH adjustor known to be safe and effective for use in injectable pharmaceutical products for the adjustment of pH may be utilized.

Although the present disclosure has been described with reference to specific embodiments, it should be understood that the limitations of the described embodiments are provided merely for purpose of illustration and are not intended to limit the present invention and associated general inventive concepts. Instead, the scope of the present invention is defined by the appended claims, and all variations and equivalents that fall within the range of the claims are intended to be embraced therein. Other embodiments than the specific exemplary ones described herein are equally possible within the scope of these appended claims. Therefore, the present invention, in its broader aspects, is not limited to the specific details, the representative compounds, and illustrative examples shown and described herein as departures may be made from such details without departing from the spirit or scope of the applicant's general inventive concept.

What is claimed is:

1. A pharmaceutical composition for the treatment of a medical condition consisting of opioid addiction, depression, opioid withdrawal side effect treatment, opioid overdose treatment without withdrawal symptoms, schizophrenia, hypertension, scleroderma, dyskinesia, bipolar disorder, ADHD, addictive behaviors, Parkinson's disease, eating disorders, anorexia nervosa, migraine, and memory disorders comprising an effective amount of the compound of formula (I):

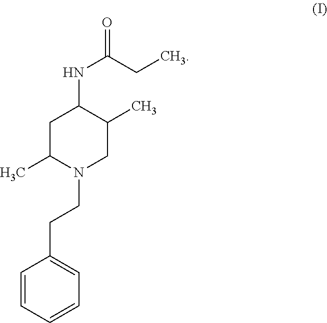 [SMILES: CCC(=O)NC1CC(C)N(CCc2ccccc2)CC1C]

2. The pharmaceutical composition of claim 1, wherein the medical condition is opioid addiction.

3. The pharmaceutical composition of claim 2, wherein the pharmaceutical composition causes no opioid withdrawal symptoms in a patient.

4. The pharmaceutical composition of claim 1, wherein the medical condition is depression.

5. The pharmaceutical composition of claim 1, wherein the medical condition is opioid withdrawal side effect treatment.

6. The pharmaceutical composition of claim 1, wherein the medical condition is schizophrenia.

7. The pharmaceutical composition of claim 1, wherein the medical condition is hypertension.

8. The pharmaceutical composition of claim 1, wherein the medical condition is scleroderma.

9. The pharmaceutical composition of claim 1, wherein the medical condition is dyskinesia associated with Parkinson's disease.

10. The pharmaceutical composition of claim 1, wherein the medical condition is eating disorder.

11. The pharmaceutical composition of claim 1, wherein the medical condition is migraine.

12. The pharmaceutical composition of claim 1, wherein the medical condition is memory disorder.

13. The pharmaceutical composition of claim 1, wherein the medical condition is opioid overdose treatment without withdrawal symptoms.

14. A method of treatment of a medical condition to an individual in need thereof wherein the medical condition consists of opioid use disorder, opioid addiction, opioid overdose, depression, opioid withdrawal side effect treatment, schizophrenia, hypertension, scleroderma, dyskinesia, bipolar disorder, ADHD, addictive behaviors, Parkinson's disease, eating disorders, anorexia nervosa, migraine, memory disorders, physiological withdrawal from opiates, comprising providing to a subject an effective amount of a pharmaceutical composition comprising the compound of formula (I):

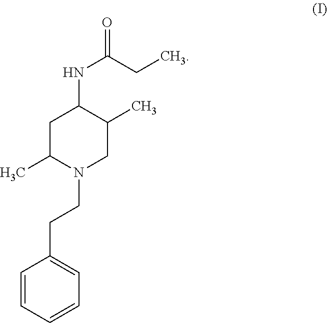 [SMILES: CCC(=O)NC1CC(C)N(CCc2ccccc2)CC1C]

15. The method according to claim 14, wherein the medical condition is opioid use disorder and the administration to the individual causes minimal withdrawal symptoms in the individual.

16. An injectable pharmaceutical composition for use in treating opioid addiction, depression, anxiety and panic disorders, hypertension, scleroderma, dyskinesia associated with Parkinson's disease, schizophrenia, bipolar disorder, ADHD, addictive behaviors, Parkinson's disease, eating disorders, anorexia nervosa, migraine, opioid overdose without withdrawal effects, and memory disorders, comprising:
(a) 1-(2-phenylethyl)-2,5-dimethyl-4-propionil aminopiperidin or a pharmaceutically acceptable salt thereof, and
(b) a pH adjustor.

17. The pharmaceutical composition according to claim 16, wherein the composition comprises:
(a) about 1 mg to about 100 mg of 1-(2-phenylethyl)-2,5-dimethyl-4-propionil aminopiperidin or a pharmaceutically acceptable salt thereof, and
(b) about 1 mg to about 100 mg of the pH adjustor.

18. The pharmaceutical composition of claim 17, wherein the 1-(2-phenylethyl)-2,5-dimethyl-4-propionil aminopiperidin or a pharmaceutically acceptable salt thereof is present in an amount of about 10 mg.

19. The pharmaceutical composition of claim 18, wherein the pH adjustor is citric acid.

20. The pharmaceutical composition of claim 19, wherein the citric acid is present in an amount of about 10 mg.

21. A pharmaceutical composition for the treatment of opioid use disorder comprising an effective amount of the compound of formula (I): 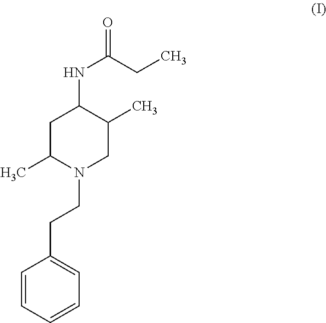 [SMILES: CCC(=O)NC1CC(C)N(CCc2ccccc2)CC1C]
wherein said treatment causes minimal withdrawal symptoms.

22. The pharmaceutical composition of claim 21, wherein the treatment of opioid use disorder comprises treatment of opioid overdose or treatment of opioid addiction.

23. The pharmaceutical composition of claim 22, wherein the patient does not require a non-dependent state for the treatment of opioid addiction.
